(19) United States
(12) Patent Application Publication
(10) Pub. No.: US 2026/0199212 A1
(43) Pub. Date: Jul. 16, 2026

(54) USE OF L-CARNOSINE AGAINST SOLAR ELASTOSIS

(71) Applicant: SYMRISE AG, Holzminden (DE)

(72) Inventors: Dominik STUHLMANN, Holzminden (DE); Maria REICHENBACH, Paderborn (DE); Paul SLAVASHEVICH, Bronx, NY (US); Diane LANFRANCHI, Yvoire (FR)

(73) Assignee: SYMRISE AG, Holzminden (DE)

(21) Appl. No.: 19/131,288

(22) PCT Filed: Nov. 25, 2022
(86) PCT No.: PCT/EP2022/083265

§ 371 Date: May 20, 2025

(51) Int. Cl. A61K 8/64; A61K 8/34; A61Q 17/04; A61Q 19/08

(52) U.S. Cl. CPC A61K 8/64; A61K 8/345; A61Q 17/04; A61Q 19/08; A61K 2800/522; A61K 2800/591

(57) ABSTRACT

The present invention relates to a non-therapeutic use of L-Carnosine for increasing the amount of fibrillin-1 in the dermis after an irradiation of the skin with infrared radiation. Furthermore, the present invention relates to a non-therapeutic use of L-Carnosine for increasing the ratio of fibrillin-1 and tropoelastin in the skin of a subject. Furthermore, the present invention relates to a non-therapeutic use of L-Carnosine for increasing the size and/or amount of elastic fibers in the skin of a subject. Furthermore, the present invention relates to a non-therapeutic use of L-Carnosine for strengthening the elastic fiber network in the skin of a subject. Furthermore, the present invention relates to a non-therapeutic use of L-Carnosine for preventing or reducing the symptoms of solar elastosis. Moreover, the present invention relates to a cosmetic composition comprising L-Carnosine.

The present invention relates to a non-therapeutic use of L-Carnosine for increasing the amount of fibrillin-1 in the dermis after an irradiation of the skin with infrared radiation. Furthermore, the present invention relates to a non-therapeutic use of L-Carnosine for increasing the ratio of fibrillin-1 and tropoelastin in the skin of a subject. Furthermore, the present invention relates to a non-therapeutic use of L-Carnosine for increasing the size and/or amount of elastic fibers in the skin of a subject. Furthermore, the present invention relates to a non-therapeutic use of L-Carnosine for strengthening the elastic fiber network in the skin of a subject. Furthermore, the present invention relates to a non-therapeutic use of L-Carnosine for preventing or reducing the symptoms of solar elastosis. Moreover, the present invention relates to a cosmetic composition comprising L-Carnosine.

Elastic fibers are an essential component of the extracellular matrix in the skin. Elastic fibers are composed of bundles of elastin, which are produced by a number of different cell types including fibroblasts.

Elastin is the major component (90%) of the elastic fibers of the skin (Ross et al., 1973. J Histochem Cytochem. 21:199-208; Ross and Bornstein, 1969. JBC. 2:366-381). It is a protein of the connective tissue responsible for the elastic properties of the skin.

Tropoelastin is the soluble monomeric precursor to elastic fibers. Following excretion from the cell, tropoelastin self associates into ~200 nm particles by coacervation. These particles continue to grow and are deposited onto fibrillin microfibrillar scaffolds.

Fibrillin is a glycoprotein, which is essential for the formation of elastic fibers, as described above. Fibrillin is secreted into the extracellular matrix by fibroblasts and forms microfibrils, which then provide a scaffold for deposition of (tropo) elastin.

Fibrillin-1 is a 340 kDa calcium-binding glycoprotein and a key component of the dermal elastic network presenting a highly complex molecular organization. In the skin, fibrillin-1 is produced by keratinocytes and fibroblasts (Haynes et al., 1997; British Journal of Dermatology. 137:17-23; Kielty and Shuttleworth, 1993. Journal of Cell Science. 106:167-173).

Solar elastosis is a cosmetic condition, which describes wrinkled skin as a result of pro-longed and excessive sun exposure of a subject. It has been found that in solar elastosis, abnormal material of the elastic tissue accumulates in the skin of a subject. Particularly, abnormal elastin fibers are found in these accumulates, which are primarily located in the dermis of the skin of a subject.

It is postulated that this accumulation of abnormal elastin fibers may be associated with increased elastin production in photodamaged skin. Furthermore, it was found that pro-longed and excessive sun exposure leads to an increased expression of the tropoelastin gene, which may contribute to increased elastin production in photodamaged skin and the accumulation of abnormal elastin fibers. These abnormal fibers are dysfunctional compared to elastin fibers with properly deposited elastin and fibrillin-1. Moreover, increased fibrillin expression and deposition have been reported in photodamaged skin.

However, a detailed study, Chen et al., Heat Modulation of Tropoelastin, Fibrillin-1, and Matrix Metalloproteinase-12 in Human Skin In Vivo, THE JOURNAL OF INVESTIGATIVE DERMATOLOGY, 124:1 Jan. 2005 reveals that tropoelastin mRNA and protein expression were increased by heat in the epidermis and dermis. Yet, it was also found that fibrillin-1 mRNA and protein expression were increased by heat in the epidermis, but were decreased in the dermis. It is suggested that this abnormal production of tropoelastin and fibrillin-1 and in particular the abnormal ratio of tropoelastin and fibrillin-1 contributes to the accumulation of abnormal elastin in the dermis and thus to the formation of solar elastosis.

It is reported in Chen et al., that heat induced an increased tropoelastin protein synthesis and that this increased tropoelastin production results in higher amounts of tropoelastin to be deposited on the fibrillin microfibrillar scaffolds, as described above. However, at the same time, heat reduced the total amount of fibrillin-1, particularly in the dermis, which leads to a reduction of microfibrils, onto which the tropoelastin needs to be deposited. This process leads to dysfunctional elastin fibers.

Consequently, a reduced ratio of fibrillin-1 and tropoelastin is observed in the skin of subjects suffering from solar elastosis.

Several treatment options for solar elastosis have been provided, such as dermabrasion, which is the surgical, controlled deeper abrasion of the upper to mid layers of the skin with any variety of strong abrasive devices including a wire brush, diamond wheel or fraise, sterilized sandpaper, salt crystals, or other mechanical means. However, such a surgical process should only be the last option for treatment.

Furthermore, active substances such as hyaluronic acid may be injected into the dermis. However, this option requires injections, which are frequently rejected by subjects, particularly, since the symptoms of solar elastosis are of cosmetic nature only.

Moreover, further active substances may also be topically applied. However, these treatments and the treatments described above have a variable efficacy.

There is thus a need for restoring a proper elastic fiber network and thus to prevent or reduce the symptoms of solar elastosis, however wherein the above disadvantages of the current options is reduced or avoided.

Thus, the primary object of the present invention is to provide an improved possibility for restoring a proper elastic fiber network and thus to prevent or reduce the symptoms of solar elastosis.

The primary object of the present invention is solved by a non-therapeutic use of L-Carnosine for increasing the amount of fibrillin-1 in the dermis after an irradiation of the skin with infrared radiation.

It was surprisingly found that L-Carnosine was able to strongly increase the amount of fibrillin-1 in the dermis after infrared irradiation. Simultaneously, it was surprisingly found that when applying L-Carnosine, the amounts of elastin were not significantly different after infrared irradiation than without infrared irradiation.

Therefore, the present invention also relates to a non-therapeutic use of L-Carnosine for increasing the ratio of fibrillin-1 and tropoelastin in the skin of a subject, preferably in the dermis of a subject, after an irradiation of the skin with infrared radiation.

The amount of fibrillin-1 and/or tropoelastin may be determined by standard methods for determining the amount of nucleic acids or proteins in a sample. Such methods are well-known to a skilled person. Preferably, the term “amount” refers to the amount of protein in this context.

Preferably, the term “ratio” refers to the weight ratio. Preferably, the term “ratio” refers to the ratio with regard to amount of substance.

The term “irradiation”, as used herein, includes the exposure of e.g. the skin to natural sunlight or the exposure to artificial light, such as IR-lamps. Preferably, artificial light provides a radiation with an intensity comparable to natural sunlight. Thus, it is preferred that artificial light, such as IR-lamps, provide infrared irradiation with an intensity in the range of from 10 to 200 mW/cm², preferably in the range of from 15 to 150 mW/cm².

As described above, infrared irradiation (heat) leads to the formation of abnormal elastic fibers. Thus, the size and the amount of proper elastic fibers are reduced after infrared irradiation.

Preferably, the term “skin cell”, as used herein, refers to skin cells, including fibroblasts and keratinocytes, in vivo, i.e. in the skin of a subject, or in vitro, i.e. in culture as a cell culture or a biopsy, each as described herein.

Preferably, the term “elastic fiber” refers to fibers comprising an elastin core, preferably an amorphous elastin core, surrounded by one or more glycosaminoglycan, preferably heparan sulphate, and one or more proteins selected from microfibrillar-associated glycoproteins, fibrillin, fibrillin, and the elastin receptor.

It was surprisingly found that this reduced size and amount of elastic fibers could be re-versed by L-Carnosine. It was surprisingly found that the increased amounts of fibrillin-1 in the dermis resulted in an increased amount of proper elastic fibers, since more fibrillin-1 microfibrils were present onto which tropoelastin could be deposited. This effect resulted in an increased size and amount of elastic fibers.

Therefore, the present invention also relates to a non-therapeutic use of L-Carnosine for increasing the size and/or amount of elastic fibers in the skin of a subject, after an irradiation of the skin with infrared radiation.

The size and amount of elastic fibers can be determined by well-known methods such as visual or software-based image analysis of microscopic images of tissue stainings, preferably wherein images are taken with confocal laser scanning microscopy.

Likewise, the increased amounts of fibrillin-1 in the dermis and the increased amount of proper elastic fibers resulted in an increased connectivity of the elastic fibers and an increased organization of the elastic fibers in the connective tissue in the skin, after these parameters were reduced by infrared irradiation.

Therefore, the present invention also relates to a non-therapeutic use for strengthening the elastic fiber network in the skin of a subject, after an irradiation of the skin with infrared radiation.

A strengthened elastic fiber network in the skin provides an increased resistance to stretch forces in the tissue, which can be determined by atomic force microscopy of skin biopsies or measurements with a cutometer.

Furthermore, a strengthened elastic fiber network is represented by an increased organization of the elastic fibers in the connective tissue, which can be determined e.g. by visual or software-based image analysis of microscopic images of tissue stainings, preferably wherein images are taken with confocal laser scanning microscopy.

The term “increasing” as used in the context of the non-therapeutic uses according to the invention preferably describes a comparison of a tissue or tissue sample, to which L-Carnosine has been applied, with the untreated control. Preferably, the term describes a comparison of subjects suffering from solar elastosis before and after L-Carnosine has been applied.

As described above, abnormal elastin fibers are found in dermal accumulates of subjects suffering from solar elastosis. Typically the symptoms of solar elastosis are or include wrin-kle formation. As described above, it was found that L-Carnosine helps restoring a proper production of elastic fibers and thus also reduces wrinkles.

Therefore, the present invention further relates to a non-therapeutic use of L-Carnosine for preventing or reducing the symptoms of solar elastosis, wherein the symptom(s) of solar elastosis prevented or reduced is or includes wrinkled skin.

The term “preventing or reducing” wrinkled skin preferably refers to the number, length and/or depth of skin wrinkles. The number and length of wrinkles may be determined by counting and measuring in images taken from the subject or on the subject directly. The depth of skin wrinkles may be determined by software-based analysis of high resolution images, for example by using the VAM software, e.g. version 5.9.7, Canfield Scientific Inc, or by Fast Optical in vivo Topometry of human skin in high definition (FOITSHD) or by using the PRIMOSCR system, Canfield Scientific Inc.

L-Carnosine may be applied to the skin cells or the skin of a subject in any possible manner. However, typically, L-Carnosine is applied topically to the skin of the subject. The skin of a subject is composed of different layers, wherein the outer layers of the skin provide a very weak permeability for L-Carnosine. Thus, substances for enhancing the penetration of L-Carnosine into the skin are frequently applied. Such substances are well-known to a skilled person and an enhancement of penetration can be measured by determining the depth of penetration of L-Carnosine, e.g. by visual, microscopic inspection of tissue stainings, particularly obtained from skin biopsies as described herein.

It is thus preferred that in the non-therapeutic uses according to the invention, L-Carnosine is topically applied to the skin of a subject, or, respectively, to a skin biopsy, wherein L-Carnosine is applied together with a substance enhancing the penetration of L-Carnosine into the skin or, respectively, skin biopsy, preferably into the dermis.

It is further preferred that the use is an in vitro use and wherein the skin of a subject refers to a skin biopsy of a subject, preferably wherein L-Carnosine is topically applied to the skin biopsy, wherein L-Carnosine is applied together with a substance enhancing the penetration of L-Carnosine into the skin biopsy, preferably into the dermis.

The term “skin biopsy” as used herein, preferably refers to removed cells or part of the skin of a subject. Such a skin biopsy may be obtained by shaving and collecting the top layers of skin (epidermis and a portion of the dermis). Further, such a skin biopsy may be obtained by a punching tool to remove a small core of skin, including deeper layers (epidermis, dermis and superficial fat). Further, such a skin biopsy may be obtained by cutting with a knife or a similar tool (e.g. scalpel) to remove an entire area of the skin, including a portion of skin down to or through the fatty layer of skin.

Typically any substance for enhancing the penetration of L-Carnosine into the skin or, respectively, skin biopsy, may be used. However, it was found that 1,2-hexane diol, 1,2-heptane diol, 1,3-propandiol, pentylene glycol, butylene glycol, propylene glycol, ethoxydiglycol, dipropylene glycol, isopropyl myristate, dimethyl isosorbide, ethanol, caprylyl glycol, and phenoxyethanol provided a particularly advantageous penetration of L-Carnosine.

Therefore, it is preferred in the non-therapeutic uses according to the invention that the, one, two, three or more or all substance(s) enhancing the penetration of L-Carnosine into the skin or, respectively, skin biopsy, preferably into the dermis is selected from the group consisting of 1,2-hexane diol, 1,2-heptane diol, 1,3-propandiol, pentylene glycol, butylene glycol, propylene glycol, ethoxydiglycol, dipropylene glycol, isopropyl myristate, dimethyl isosorbide, ethanol, caprylyl glycol, phenoxyethanol, and combinations thereof.

It is preferred that L-Carnosine is applied in a cosmetic formulation.

Different concentrations of L-Carnosine have been tested, wherein the higher concentrations provided a stronger effect.

It is thus preferred that L-Carnosine is applied in a cosmetic formulation, wherein the cosmetic formulation comprises L-Carnosine in amounts in the range of from 0.05 wt.-% to 10 wt.-%, preferably 0.075 to 7.5 wt.-%, preferably 0.1 wt.-% to 5 wt.-%, preferably 0.125 to 4 wt.-%, preferably 0.15 to 3 wt.-%; preferably 0.175 to 2.5 wt.-%, based on the total weight of the cosmetic formulation, preferably on the total weight of the dry matter of the cosmetic formulation.

It was surprisingly found that an amount of at least 0.2 wt.-% L-Carnosine, based on the total weight of the cosmetic formulation, preferably on the total weight of the dry matter of the cosmetic formulation, has provided particularly advantageous results.

It is thus preferred that L-Carnosine is applied in a cosmetic formulation, wherein the cosmetic formulation comprises L-Carnosine in amounts in the range of from 0.15 wt.-% to 10 wt.-%, preferably 0.15 to 7.5 wt.-%, preferably 0.15 wt.-% to 5 wt.-%, preferably 0.175 to 4 wt.-%, preferably 0.175 to 3 wt.-%; preferably 0.175 to 2.5 wt.-%, preferably at least 0.15 wt.-%, preferably at least 0.175 wt.-%, preferably at least 0.2 wt.-%, based on the total weight of the cosmetic formulation, preferably on the total weight of the dry matter of the cosmetic formulation.

Furthermore, it is preferred that the cosmetic formulation is applied such that the L-Carnosine is applied in an amount in the range of from 1 to 7.5 μg per cm² skin surface or, respectively, surface of the skin biopsy.

It was found that L-Carnosine may be applied to prevent the negative effects of infrared irradiation. Thus, the L-Carnosine can be applied before the irradiation.

It is thus preferred that the L-Carnosine is applied to the skin or, respectively, skin biopsy before exposure of the skin or, respectively, skin biopsy to the infrared irradiation, preferably in a range of from 1 min to 48 hours, preferably in a range of from 2 min to 24 hours, preferably in a range of from 5 min to 12 hours, preferably in a range of from 10 min to 6 hours, preferably in a range of from 15 min to 4 hours, preferably in a range of from 20 min to 2 hours, preferably in a range of from 30 min to 1 hour, before the exposure.

Similarly, it was also found that L-Carnosine may be applied to reduce the negative effects of infrared irradiation. Thus, the L-Carnosine can be applied after the irradiation.

It is thus preferred that the L-Carnosine is applied to the skin or, respectively, skin biopsy after exposure of the skin or, respectively, skin biopsy to the infrared irradiation,

preferably in a range of from 1 min to 48 hours, preferably in a range of from 2 min to 24 hours, preferably in a range of from 5 min to 12 hours, preferably in a range of from 10 min to 6 hours, preferably in a range of from 15 min to 4 hours, preferably in a range of from 20 min to 2 hours, preferably in a range of from 30 min to 1 hour, after the exposure.

Furthermore, the present invention relates to a cosmetic composition comprising
(i) L-Carnosine, and
(ii) one, two, three or more substances enhancing the penetration of L-Carnosine into the skin or, respectively, skin biopsy,

wherein the amount of L-Carnosine in the composition is sufficient for increasing the amount of fibrillin-1 in the dermis after an irradiation of the skin with infrared radiation, and/or
increasing the ratio of fibrillin-1 and tropoelastin in the skin of a subject, preferably in the dermis of a subject, after an irradiation of the skin with infrared radiation, and/or
increasing the size and/or amount of elastic fibers in the skin of a subject, after an irradiation of the skin with infrared radiation, and/or
strengthening the elastic fiber network in the skin of a subject, after an irradiation of the skin with infrared radiation, and/or
preventing or reducing the symptoms of solar elastosis, wherein the symptom(s) of solar elastosis prevented or reduced is or includes wrinkled skin.

Advantageously such a composition can be used for performing the non-therapeutic uses according to the invention.

For determining whether the respective increase, strengthening, prevention or reduction is obtained by the amount of L-Carnosine in the composition, the composition can be applied and it can be determined whether the corresponding effect is obtained or not, as described herein for the non-therapeutic uses.

Furthermore, for determining whether the respective increase, strengthening, prevention or reduction is obtained by the amount of L-Carnosine in the composition, an in vitro approach may be performed, in which skin cells or a skin biopsy is provided and cultivated and in which the respective parameters are measured. Subsequently, the respective amount of L-Carnosine (or respectively a composition comprising the respective amount of L-Carnosine) is administered and the skin cells or skin biopsy is irradiated with infrared radiation. Afterwards, the above parameters are measured again and compared with the first measurement.

It is thus preferred that the sufficient amount of L-Carnosine in the cosmetic composition describes an amount, which causes the respective increase, strengthening, prevention or reduction when performing the following in vitro assay:
i) providing and cultivating a cell culture comprising skin cells or a skin biopsy, ii) measuring the amount of fibrillin-1 and/or tropoelastin in the cell culture or in the skin biopsy, and/or
   measuring the size and/or amount of elastic fibers in the skin biopsy, and/or
   measuring the connectivity of the elastic fibers in the skin biopsy, and/or
   measuring the amount of fibrillin-1 and/or tropoelastin in the cell culture or in the skin biopsy and determining the ratio of fibrillin-1 and tropoelastin, and/or
   measuring the number, length and/or depth of skin wrinkles in the skin biopsy,
iii) administering a pre-determined amount of L-Carnosine to the cell culture or skin biopsy,
iv) irradiating the skin cells or skin biopsy,
   preferably with infrared radiation with an irradiance in the range of from 10 to 200 mW/cm², preferably in the range of from 15 to 150 mW/cm², preferably with infrared radiation with an irradiance in the range of from 500 to 800 J/cm², preferably in the range of from 600 to 750 J/cm²,
v) repeating step ii) and comparing the obtained measurement to the measurement obtained in step ii).

Preferably, step iv) of the in vitro approach is performed after step iii) of the in vitro approach. Preferably, step iv) of the in vitro approach is performed before step iii) of the in vitro approach.

Preferably, the term “cell culture”, as used herein, refers to a two-dimensional cell culture comprising the respective cells, whereas the term “skin biopsy” preferably refers to a three-dimensional culture, which is obtained from a subject and cultivated.

Furthermore, it is preferred in the cosmetic composition according to the invention that the, one, two, three or more or all substances enhancing the penetration of L-Carnosine into the skin or, respectively, skin biopsy, is selected from the group consisting of 1,2-hexane diol, 1,2-heptane diol, 1,3-propandiol, pentylene glycol, butylene glycol, propylene glycol, ethoxydiglycol, dipropylene glycol, isopropyl myristate, dimethyl isosorbide, ethanol, caprylyl glycol, phenoxyethanol, and combinations thereof.

Preferably, the terms “increasing”, “strengthening” and “reducing” describe a difference of the respective parameter between the untreated and the treated sample or, respectively, before and after the treatment, of at least 5%, preferably at least 7.5%, preferably at least 10%, preferably at least 12.5%, preferably at least 15%, preferably at least 20%, based on the untreated sample or, respectively, on the measurement before the treatment.

Moreover, the present invention relates to a method for increasing the amount of fibrillin-1 in the dermis after an irradiation of the skin cells with infrared radiation, comprising the steps
i) identifying a patient in need of increasing the amount of fibrillin-1 in the dermis, and
ii) administering L-Carnosine to the skin cells.

Moreover, the present invention relates to a method for increasing the ratio of fibrillin-1 and tropoelastin in the skin of a subject, preferably in the dermis, after an irradiation of the skin with infrared radiation, comprising the steps
i) identifying a patient in need of increasing the ratio of fibrillin-1 and tropoelastin in the skin, preferably in the dermis, and
ii) administering L-Carnosine to the skin cells.

Moreover, the present invention relates to a method for increasing the size and/or amount of elastic fibers in the skin of a subject, after an irradiation of the skin with infrared radiation, comprising the steps
i) identifying a patient in need of increasing the size and/or amount of elastic fibers in the skin, and
ii) administering L-Carnosine to the skin cells.

Moreover, the present invention relates to a method for strengthening the elastic fiber network in the skin of a subject, after an irradiation of the skin with infrared radiation, comprising the steps
i) identifying a patient in need of strengthening the elastic fiber network in the skin, and
ii) administering L-Carnosine to the skin cells.

Moreover, the present invention relates to a method for preventing or reducing the symptoms of solar elastosis, comprising the steps
i) identifying a patient in need of preventing or reducing the symptoms of solar elastosis, and
ii) administering L-Carnosine to the skin cells, wherein the symptom(s) of solar elastosis prevented or reduced is or includes wrinkled skin.

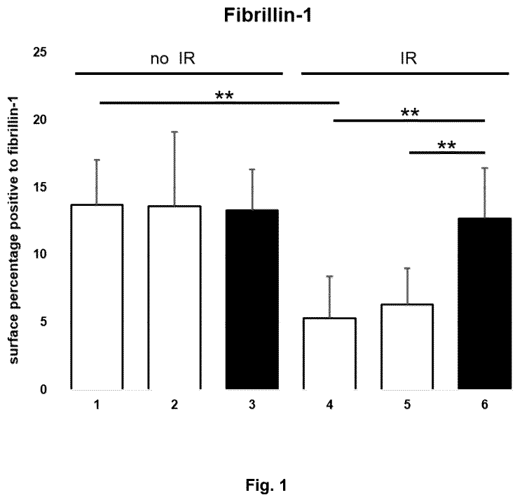

FIG. 1 shows the surface percentage positive to fibrillin-1 according to Example 2.2, with the non-treated control (1; batch C1), the treatment with composition A (2; batch C2), the treatment with composition B (3; batch T), non-treated control after IR-irradiation (4; batch IR), the treatment with composition A after IR-irradiation (5; batch IR-C2), the treatment with composition B after IR-irradiation (6; batch IR-T), error bars represent the standard deviation, with **: p<0.01.

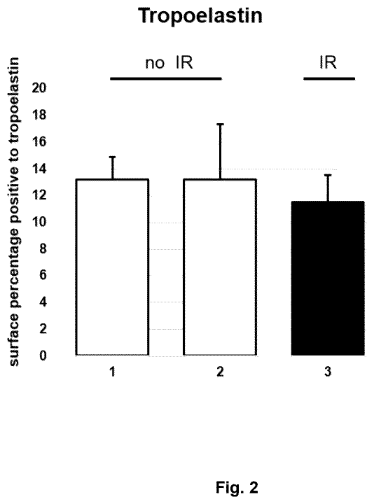

FIG. 2 shows the surface percentage positive to tropoelastin according to Example 2.3, with the non-treated control (1; batch C1), the treatment with composition B (2; batch T) and the treatment with composition B after IR-irradiation (3; batch IR-T), error bars represent the standard deviation.

Further aspects and advantages of the invention result from the subsequent description of preferred examples.

### EXAMPLES

### Example 1: Study Conditions

### Example 1.1: Tested Compositions

The following compositions were provided and tested in the following study.

### Composition a (Comparison)

| | | Amount |
|---|---|---|
| Phase | Compound | [wt.-%] |
| A | Water | 86.56 |
| | Phenoxyethanol, Decylene glycol and 1,2-Hexanediol | 1.00 |
| | Acrylates/C10-30 Alkyl acrylate crosspolymer | 0.05 |
| B | Acrylates/C10-30 Alkyl acrylate crosspolymer | 0.20 |
| C | Cetearyl alcohol | 2.00 |
| | Caprylic/Capric triglyceride | 10.00 |
| D | Water, CITRIC ACID (33%) | 0.19 |

### Composition B (According to the Invention)

| | | Amount |
|---|---|---|
| Phase | Compound | [wt.-%] |
| A | Water | 86.36 |
| | Phenoxyethanol, Decylene glycol and 1,2-Hexanediol | 1.00 |
| | Acrylates/C10-30 Alkyl acrylate crosspolymer | 0.05 |
| B | Acrylates/C10-30 Alkyl acrylate crosspolymer | 0.20 |
| C | Cetearyl alcohol | 2.00 |
| | Caprylic/Capric triglyceride | 10.00 |
| D | Water, CITRIC ACID (33%) | 0.19 |
| E | L-Carnosine | 0.2 |

The compositions were provided as follows:

Sprinkle the polymer in phase A and let it swell, then heat to 80° C. Heat phase C to 80° C. Disperse phase B into phase A and add C into the mixture of A and B under high shear. Neutralize with phase D and let cool down. Where applicable: At a temperature<40° C., add phase E. Set the pH to 6.

The compositions have been stored at room temperature before, within and after the period of the study.

### Example 1.2: Skin Samples

Human skin explants of an average diameter of 12 mm (+1 mm) were prepared on an abdo-plasty coming from a 54-year-old caucasian woman with a phototype II according to Fitz-patrick skin colour classification. The explants were kept in survival in BEM culture medium (BIO-EC's Explants Medium) at 37° C. in a humid, 5%-CO2 atmosphere.

Skin samples were distributed into batches as follows

| | | Number of | Sampling |
|---|---|---|---|
| Batch | Designation | samples | time |
| C0 | Tissue control | 3 | Day 0 |
| C1 | Non-treated control | 3 | Day 7 |
| C2 | Composition A | 3 | Day 7 |
| T | Composition B | 3 | Day 7 |
| IR | IR-irradiation | 3 | Day 7 |
| IR-C2 | IR-irradiation, Composition A | 3 | Day 7 |
| IR-T | IR-irradiation, Composition B | 3 | Day 7 |

Compositions A and B were applied at the rate of 2 μL per 1 cm² skin sample (~2 mg/cm²) and spread using a small spatula on day 0, day 1, day 2, day 3 and day 6 (30 min before infrared irradiation exposure). The control samples C0 and C1 did not receive any treatment except the renewal of culture medium. The culture medium was half renewed (1 ml per well) on day 2, day 3 and day 6.

### Example 1.3: Irradiation

On day 6, the culture media of all the batches was replaced by HBSS (Hank's Balanced Saline Solution; 1 ml per explant). The batches IR, IR-C2, and IR-T were irradiated using a infrared lamp (Dr FISCHER 1000W, 235V 2500K; 760-3000 nm), for 1:24 h (720 J/cm²; 140 mW/cm²). The non-irradiated batches were kept in HBSS in the dark. At the end of the irradiation, all the explants were put back in 2 mL of fresh BEM medium.

On day 0, the 3 samples of the batch C0 were collected and cut in two parts. One half was fixed in buffered formalin and the other half was frozen at −80° C. On day 7, 24 h after the exposure to infrared, the 3 samples from each batch were collected and treated the same way as the samples of C0 on day 0.

### Example 1.4: Histological Processing

After fixation for 24 hours in buffered formalin, the samples were dehydrated and impreg-nated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The microscopical observations were realized using a Leica DMLB, an Olympus BX43 or BX63 microscope. Pictures were digitized with a numeric DP72 or DP74 Olympus camera with cellSens storing software.

### Cell Viability Analysis:

The cell viability of epidermal and dermal structures was observed on formol-fixed paraffine embedded (FFPE) skin sections after Masson's trichrome staining, Goldner variant. The cellular viability was assessed by microscopical observation. Concerned batches: all.

### Fibrillin-1 Immunostaining:

Fibrillin-1 was stained on frozen skin sections with a polyclonal anti-fibrillin-1 antibody (Sigma Aldrich, ref. HPA021057), diluted at 1:200 in PBS-BSA 0.3%, incubated over night at room temperature and revealed using AF488 (Life technologies, A11008). Nuclei have been post-stained with propidium iodide. The staining was assessed by microscopical observation and semi-quantified by image analysis. Concerned batches: all.

### Tropoelastin Immunostaining:

Tropoelastin was stained on frozen skin sections with a monoclonal anti-elastin antibody (Chemicon, ref. MAB2503), diluted at 1:50 in PBS-BSA 0.3%, incubated over night at room temperature and revealed using AF488 (Life technologies, A11001). Nuclei have been post stained with propidium iodide. The staining was assessed by microscopical observation and semi-quantified by image analysis. Concerned batches: all.

### Image Analysis:

Fibrillin-1 immunostaining was assessed by microscopical observation of skin sections with semi-quantification by image analysis using the software cellSens (Olympus).

Concerned batches: all.

Analysed zone: ROI including the papillary dermis.

Number of analysed images per batch: 9.

Statistical test: Student's T test.

Tropoelastin immunostaining was assessed by microscopical observation of skin sections with semi-quantification by image analysis using the software cellSens (Olympus).

Concerned batches: all.

Analysed zone: ROI including the papillary dermis.

Number of analysed images per batch: 9.

Statistical test: Student's T test.

### Example 2: Study Results

### Example 2.1: Cell Viability

The cell viability of the different batches was determined as described in Example 1.4. The following results were obtained:

Cell viability

| Batch | Epidermis | Dermis |
|---|---|---|
| C0 | G | G |
| C1 | FG | G |
| C2 | SA | G |
| T | FG | G |
| IR | FG | G |
| IR-C2 | VSA | G |
| IR-T | VSA | G |

Morphology legend: G = good, FG = fairly good, VSA = very slightly altered, SA = slightly altered, MA = moderately altered, FCA = fairly clearly altered, CA = clearly altered, VCA = very clearly altered

In the epidermis, no irradiation:

On day 0, on the blank batch (C0), the cell viability was good. On day 7, on the blank batch (C1), the cell viability was fairly good.

### Compared to C1:

Composition A (C2) induced slight epidermal alterations, composition B (T) induced no modification.

### Compared to C2:

Composition B (T) induced a slight improvement.

In the Epidermis, with Irradiation:

### Comparing IR to C1:

The infrared irradiation induced no modification of the cell viability.

### Compared to IR:

Composition A (C2) induced very slight epidermal alterations. Composition B (T) induced very slight epidermal alterations.

### Compared to C2:

Composition B (T) induced no modification.

### In the Dermis:

No alteration was detected in any of the samples.

### Conclusion:

The tested conditions and applied treatments did not reduce the cell viability in the dermis, only slight modifications were observed in the epidermis.

### Example 2.2: Fibrillin-1

The percentage of surface positive to fibrillin-1 immunostaining in the papillary dermis of the different batches was determined as described in Example 1.4. The following results (including the standard deviation, SD) were obtained:

Fibrillin-1

| Batch | (% surface, mean) | SD |
|---|---|---|
| C0 | 7.4 | 3.1 |
| C1 | 13.7 | 3.3 |
| C2 | 13.6 | 5.5 |
| T | 13.3 | 3.0 |
| IR | 5.3 | 3.1 |
| IR-C2 | 6.3 | 2.7 |
| IR-T | 12.7 | 3.7 |

The results are also depicted in FIG. 1.

### Comparing IR to C1:

The infrared irradiation induced a significant decrease of fibrillin-1 expression in the papillary dermis by 61%** (with **: p<0.01).

### Compared to IR:

Composition A did not lead to a significant increase of fibrillin-1 expression. However, composition B induced a significant increase of 140%** (with **: p<0.01).

### Compared to IR-C2:

Composition B induced a significant increase of 101%** (with **: p<0.01).

### Example 2.3: Tropoelastin

The percentage of surface positive to tropoelastin immunostaining in the papillary dermis of the different batches was determined as described in Example 1.4. The following results (including the standard deviation, SD) were obtained:

Tropoelastin

| Batch | (% surface, mean) | SD |
|---|---|---|
| C1 | 13.2 | 1.7 |
| T | 13.2 | 4.1 |
| IR-T | 11.5 | 2.0 |

No significant differences were observed between the untreated control (C1), the sample treated with composition B (T) and the sample irradiated and treated with composition B (IR-T).

The results are also depicted in FIG. 2.

1-15. (canceled)

16. A method for treating skin of a subject, the method comprising applying a composition comprising L-carnosine to the skin before or after exposure of the skin to infrared radiation.

17. The method of claim 16, wherein the method increases the amount of fibrillin-1 in the dermis of the skin.

18. The method of claim 16, wherein the method increases the ratio of fibrillin-1 to tropoelastin in the skin.

19. The method of claim 16, wherein the method increases the size and/or amount of elastic fibers in the skin.

20. The method of claim 16, wherein the method strengthens the elastic fiber network in the skin.

21. The method of claim 16, wherein the method prevents or reduces one or more symptoms of solar elastosis, wherein at least one symptom that is prevented or reduced is skin wrinkling.

22. The method of claim 16, wherein the L-carnosine is topically applied to the skin with one or more substances that enhance penetration of the L-carnosine into the skin.

23. The method of claim 16, wherein the skin is a skin biopsy of the subject.

24. The method of claim 22, wherein the one or more substances that enhance penetration of the L-carnosine into the skin are selected from 1,2-hexane diol, 1,2-heptane diol, 1,3-propandiol, pentylene glycol, butylene glycol, propylene glycol, ethoxydiglycol, dipropylene glycol, isopropyl myristate, dimethyl isosorbide, ethanol, caprylyl glycol, and phenoxyethanol.

25. The method of claim 22, wherein the L-carnosine is topically applied to the skin in a cosmetic formulation comprising 0.05 to 10 wt. % of the L-carnosine, based on a total weight of the cosmetic formulation.

26. The method of claim 25, wherein the L-carnosine is applied to the skin in an amount of 1 to 7.5 μg per cm² skin surface.

27. The method of claim 16, wherein the L-carnosine is applied to the skin before exposure of the skin to infrared radiation.

28. The method of claim 27 wherein the L-carnosine is applied to the skin 1 minute to 48 hours before exposure of the skin to infrared radiation.

29. The method of claim 16, wherein the L-carnosine is applied to the skin after exposure of the skin to infrared radiation.

30. The method of claim 29, wherein the L-carnosine is applied to the skin 1 minute to 48 hours after exposure of the skin to infrared radiation.

31. A cosmetic composition comprising:
(i) L-carnosine; and
(ii) one or more substances that enhance penetration of the L-carnosine into skin;
wherein the composition contains an amount of L-carnosine effective for:
increasing fibrillin-1 level in dermis after exposure of the skin to infrared radiation;
increasing the ratio of fibrillin-1 to tropoelastin in the skin after infrared irradiation;
increasing the size and/or quantity of elastic fibers in the skin after infrared irradiation;
strengthening the elastic fiber network in the skin following infrared radiation; and/or
preventing or reducing one or more symptoms of solar elastosis, wherein at least one symptom that is prevented or reduced is skin wrinkling.

32. The cosmetic composition of claim 31, wherein the effective amount of L-carnosine is defined as an amount that produces the respective increase, strengthening, prevention, or reduction as determined by the following in vitro assay:
(i) providing and cultivating a cell culture comprising skin cells or a skin biopsy;
(ii) performing one or more of the following measurements on the cell culture or skin biopsy:
measuring the amount of fibrillin-1 and/or tropoelastin;
measuring the size and/or amount of elastic fibers;
measuring the connectivity of elastic fibers;
determining the ratio of fibrillin-1 to tropoelastin; and/or
measuring the number, length, and/or depth of skin wrinkles;
(iii) administering a predetermined amount of L-carnosine to the cell culture or skin biopsy;
(iv) irradiating the skin cells or skin biopsy with:
an irradiance in the range of 10 to 200 mW/cm²; and/or
an energy dose in the range of 500 to 800 J/cm²; and
v) repeating step (ii) and comparing the resulting measurements with those obtained in initial step (ii).

33. The cosmetic composition of claim 31, wherein the one or more substances that enhance the penetration of the L-carnosine into the skin are selected from 1,2-hexanediol, 1,2-heptanediol, 1,3-propanediol, pentylene glycol, butylene glycol, propylene glycol, ethoxydiglycol, dipropylene glycol, isopropyl myristate, dimethyl isosorbide, ethanol, caprylyl glycol, and phenoxyethanol.
